(19) United States
(12) Patent Application Publication
(10) Pub. No.: US 2008/0188494 A1
(43) Pub. Date: Aug. 7, 2008

(54) Use Of 5-Alkyl-6-Phenylalkyl-7-Aminoazolopyrimidines, Novel Azolopyrimidines, Processes For Their Preparation And Compositions Comprising Them

(75) Inventors: Jochen Dietz, Mannheim (DE); Wassilios Grammenos, Ludwigshafen (DE); Thomas Grote, Wachenheim (DE); Udo Hunger, Mannheim (DE); Jan Klaas Lohmann, Mannheim (DE); Bernd Muller, Frankenthal (DE); Joachim Rheinheimer, Ludwigshafen (DE); Peter Schafer, Ottersheim (DE); Frank Schieweck, Hessheim (DE); Anja Schwogler, Mannheim (DE)

Correspondence Address: HUTCHISON LAW GROUP PLLC, PO BOX 31686, RALEIGH, NC, 27612, US

(73) Assignee: BASF Aktiengesellschaft, Ludwigshafen (DE)

(21) Appl. No.: 11/912,335

(22) PCT Filed: Apr. 24, 2006
(86) PCT No.: PCT/EP2006/061786

§ 371 Date: Oct. 23, 2007

(30) Foreign Application Priority Data
Apr. 25, 2005 (DE) 102005019399.4

(51) Int. Cl. A01N 43/90; C07D 487/04; A01P 3/00

(52) U.S. Cl. 514/259.31; 544/254; 544/263; 514/259.3

(57) ABSTRACT

The use of 5-alkyl-6-phenylalkyl-7-aminoazolopyrimidines of the formula I

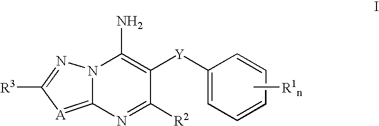

in which the variables are as defined below:
Y is alkylene, alkenylene or alkynylene, optionally substituted by alkyl groups;
R¹ is halogen, cyano, nitro, hydroxyl, mercapto, alkyl, haloalkyl, alkenyl, cycloalkyl, cycloalkenyl, alkoxy, haloalkoxy, alkenyloxy, alkynyloxy, alkylthio, NR^{A}R^{B}, alkylcarbonyl, phenyl, naphthyl, or a five- or six-membered saturated, partially unsaturated or aromatic heterocycle which contains one to four heteroatoms from the group consisting of O, N and S;
   R^{A}, R^{B} is hydrogen, alkyl or alkylcarbonyl;
n is zero, 1, 2, 3 or 4;
R² is alkyl, alkenyl, cycloalkyl, alkoxyalkyl or alkylthioalkyl;
R³ is hydrogen, halogen, cyano, NR^{A}R^{B}, hydroxyl, mercapto, alkyl, haloalkyl, cycloalkyl, alkoxy, alkylthio, cycloalkoxy, cycloalkylthio, carboxyl, formyl, alkylcarbonyl, alkoxycarbonyl, alkenyloxycarbonyl, alkynyloxycarbonyl, phenyl, phenoxy, phenylthio, benzyloxy, benzylthio, alkyl-S(O)ₘ—;
   m is 0, 1 or 2;
A is N or C—R^{a};
   R^{a} is hydrogen or alkyl;
      where the carbon atoms in Y, R¹, R², R³ and R^{a} may be substituted according to the description;
      for controlling phytopathogenic harmful fungi; novel 5-alkyl-6-phenylalkyl-7-amino-azolopyrimidines, processes for their preparation and compositions comprising them.

The present invention relates to the use of 5-alkyl-6-phenylalkyl-7-aminoazolo-pyrimidines of the formula I

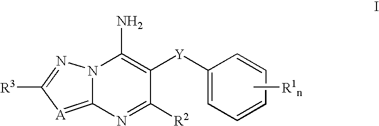

in which the variables are as defined below:
Y is C₁-C₆-alkylene, C₂-C₆-alkenylene or C₂-C₆-alkynylene, optionally substituted by 1 to 4 C₁-C₆-alkyl groups;
R¹ is halogen, cyano, nitro, hydroxyl, mercapto, C₁-C₆-alkyl, C₁-C₄-haloalkyl, C₂-C₆-alkenyl, C₃-C₆-cycloalkyl, C₃-C₆-cycloalkenyl, C₁-C₆-alkoxy, C₁-C₆-haloalkoxy, C₂-C₆-alkenyloxy, C₃-C₆-alkynyloxy, C₁-C₆-alkylthio, NR^{A}R^{B}, C₁-C₆-alkylcarbonyl, phenyl, naphthyl, or a five- or six-membered saturated, partially unsaturated or aromatic heterocycle which contains one to four heteroatoms from the group consisting of O, N and S;
   R^{A}, R^{B} are hydrogen, C₁-C₆-alkyl or C₁-C₆-alkylcarbonyl;
n is zero, 1, 2, 3 or 4;
R² is C₂-C₆-alkyl, C₂-C₄-alkenyl, C₃-C₆-cycloalkyl, C₁-C₁₂-alkoxy-C₁-C₁₂-alkyl or C₁-C₁₂-alkylthio-C₁-C₁₂-alkyl;
R³ is hydrogen, halogen, cyano, NR^{A}R^{B}, hydroxyl, mercapto, C₂-C₆-alkyl, C₁-C₆-halo-alkyl, C₃-C₈-cycloalkyl, C₁-C₆-alkoxy, C₁-C₆-alkylthio, C₃-C₈-cycloalkoxy, C₃-C₈-cycloalkylthio, carboxyl, formyl, C₁-C₁₀-alkylcarbonyl, C₁-C₁₀-alkoxycarbonyl, C₂-C₁₀-alkenyloxycarbonyl, C₂-C₁₀-alkynyloxycarbonyl, phenyl, phenoxy, phenylthio, benzyloxy, benzylthio, C₁-C₆-alkyl-S(O)ₘ—;
   m is 0, 1 or 2;
A is N or C—R^{a};
   R^{a} is hydrogen or C₁-C₆-alkyl;
      where the carbon atoms in Y, R¹, R², R³ and R^{a} may carry one to three groups R^{b}:
R^{b} is halogen, cyano, nitro, hydroxyl, C₃-C₆-cycloalkyl, C₁-C₆-alkoxy, C₁-C₆-alkylthio or NR^{A}R^{B};
   for controlling phytopathogenic harmful fungi.

Moreover, the invention relates to novel 5-phenylalkyl-6-alkyl-7-aminoazolopyrimidines, to processes for preparing these compounds and to compositions comprising them.

U.S. Pat. No. 5,389,632 and WO 02/064211 describe individual 5-alkyl-6-biphenylalkyl-7-amino-pyrazolopyrimidines having pharmaceutical action. EP-A 141 317 discloses individual 5-alkyl-6-phenylalkyl-7-aminoazolopyrimidines. The compounds from the last-mentioned document are described as being fungicidally active. However, in many cases their activity is unsatisfactory. Based on this, it is an object of the present invention to provide compounds having improved activity and/or a wider activity spectrum.

We have found that this object is achieved by the compounds defined at the outset. Furthermore, we have found processes and intermediates for their preparation, compositions comprising them and methods for controlling harmful fungi using the compounds I.

The compounds of the formula I differ from the compounds known from EP-A 141 317 essentially by the specific embodiment of the substituent in the 5-position of the azolopyrimidine skeleton.

Compared to the known compounds, the compounds of the formula I are more effective against harmful fungi.

The compounds according to the invention can be obtained by different routes. Advantageously, the compounds according to the invention are obtained by reacting substituted β-ketoesters of the formula II with a 3-aminoazole of the formula III to give 7-hydroxytriazolopyrimidines of the formula IV. The groups R¹ and R² in formulae II and IV are as defined for formula I and the group R in formula II is C₁-C₄-alkyl; for practical reasons, preference is given here to methyl, ethyl or propyl.

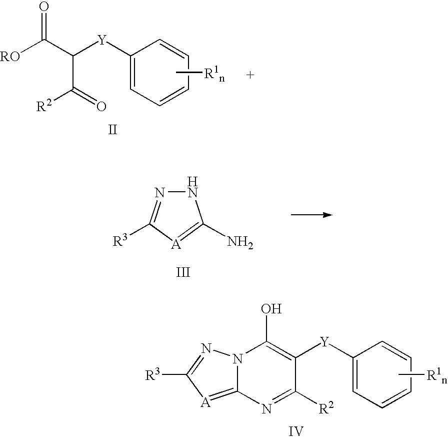

The reaction of the substituted β-ketoesters of the formula II with the aminoazoles of the formula III can be carried out in the presence or absence of solvents. It is advantageous to use solvents to which the starting materials are substantially inert and in which they are completely or partially soluble. Suitable solvents are in particular alcohols, such as ethanol, propanols, butanols, glycols or glycol monoethers, diethylene glycols or their monoethers, aromatic hydrocarbons, such as toluene, benzene or mesitylene, amides, such as dimethylformamide, diethylformamide, dibutylformamide, N,N-dimethylacetamide, lower alkanoic acids, such as formic acid, acetic acid, propionic acid, or bases, such as alkali metal and alkaline earth metal hydroxides, alkali metal and alkaline earth metal oxides, alkali metal and alkaline earth metal hydrides, alkali metal amides, alkali metal and alkaline earth metal carbonates and also alkali metal bicarbonates, organometallic compounds, in particular alkali metal alkyls, alkylmagnesium halides and also alkali metal and alkaline earth metal alkoxides and dimethoxymagnesium, moreover organic bases, for example tertiary amines, such as trimethylamine, triethylamine, triisopropylethylamine, tributylamine and N-methyl-piperidine, N-methylmorpholine, pyridine, substituted pyridines, such as collidine, lutidine and 4-dimethylaminopyridine, and also bicyclic amines and mixtures of these solvents with water. Suitable catalysts are bases as mentioned above or acids such as sulfonic acids or mineral acids. With particular preference, the reaction is carried out in the absence of a solvent or in chlorobenzene, xylene, dimethyl sulfoxide or N-methyl-pyrrolidone. Particularly preferred bases are tertiary amines, such as triisopropyl-ethylamine, tributylamine, N-methylmorpholine or N-methylpiperidine. The temperatures are from 50 to 300° C., preferably from 50 to 180° C., if the reaction is carried out in solution [cf. EP-A 770 615; Adv. Het. Chem. 57 (1993), 81ff].

The bases are generally employed in catalytic amounts; however, they can also be employed in equimolar amounts, in excess or, if appropriate, as solvents.

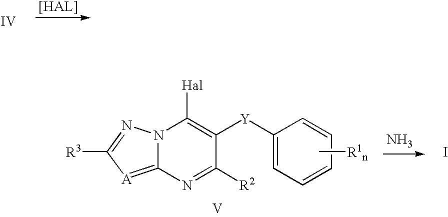

In most cases, the resulting condensates of the formula IV precipitate from the reaction solutions in pure form and, after washing with the same solvent or with water and subsequent drying, they are reacted with halogenating agents, in particular chlorinating or brominating agents, to give the compounds of the formula V in which Hal is chlorine or bromine, in particular chlorine. The reaction is preferably carried out using chlorinating agents such as phosphorus oxychloride, thionyl chloride or sulfuryl chloride at from 50° C. to 150° C., preferably in excess phosphorus oxytrichloride at reflux temperature. After evaporation of excess phosphorus oxytrichloride, the residue is treated with ice-water, if appropriate with addition of a water-immiscible solvent. In most cases, the chlorinated product isolated from the dried organic phase, if appropriate after evaporation of the inert solvent, is very pure and is subsequently reacted with ammonia in inert solvents at from 100° C. to 200° C. to give the 7-amino-triazolo[1,5-a]pyrimidines. The reaction is preferably carried out using a 1- to 10-molar excess of ammonia, under a pressure of from 1 to 100 bar.

The novel 7-aminoazolo[1,5-a]pyrimidines are, if appropriate after evaporation of the solvent, isolated as crystalline compounds by digestion in water.

The β-ketoesters of the formula II can be prepared as described in Organic Synthesis Coll. Vol. 1, p. 248, and/or they are commercially available.

Alternatively, the novel compounds of the formula I can be obtained by reacting substituted acyl cyanides of the formula VI in which R¹ and R² are as defined above with 3-amino-1,2,4-triazole of the formula III.

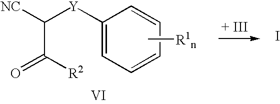

The reaction can be carried out in the presence or absence of solvents. It is advantageous to use solvents to which the starting materials are substantially inert and in which they are completely or partially soluble. Suitable solvents are in particular alcohols, such as ethanol, propanols, butanols, glycols or glycol monoethers, diethylene glycols or their monoethers, aromatic hydrocarbons, such as toluene, benzene or mesitylene, amides, such as dimethylformamide, diethylformamide, dibutylformamide, N,N-dimethylacetamide, lower alkanoic acids, such as formic acid, acetic acid, propionic acid, or bases, such as those mentioned above, and mixtures of these solvents with water. The reaction temperatures are from 50 to 300° C., preferably from 50 to 150° C., if the reaction is carried out in solution.

The novel 7-aminoazolo[1,5-a]pyrimidines of the formula I are, if appropriate after evaporation of the solvent or dilution with water, isolated as crystalline compounds.

Some of the substituted alkyl cyanides of the formula VI required for preparing the 7-aminoazolo[1,5-a]pyrimidines are known, or they can be prepared by known methods from alkyl cyanides and carboxylic acid esters using strong bases, for example alkali metal hydrides, alkali metal alkoxides, alkali metal amides or metal alkyls (cf.: J. Amer. Chem. Soc. 73, (1951), p. 3766).

If individual compounds I cannot be obtained by the routes described above, they can be prepared by derivatization of other compounds I.

If the synthesis yields mixtures of isomers, a separation is generally not necessarily required, however, since in some cases the individual isomers can be interconverted during work-up for use or during application (for example under the action of light, acids or bases). Such conversions may also take place after use, for example in the treatment of plants in the treated plant, or in the harmful fungus to be controlled.

In the definitions of symbols given in the formulae above, collective terms were used which are generally representative of the following substituents:

halogen: fluorine, chlorine, bromine and iodine;

alkyl: saturated straight-chain or mono- or dibranched hydrocarbon radicals having 1 to 4, 6, 8 or 12 carbon atoms, for example C₁-C₆-alkyl such as methyl, ethyl, propyl, 1-methylethyl, butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethyl-butyl, 2,3-dimethylbutyl, 3,3-dimethylbutyl, 1-ethylbutyl, 2-ethylbutyl, 1,1,2-trimethyl-propyl, 1,2,2-trimethylpropyl, 1-ethyl-1-methylpropyl and 1-ethyl-2-methylpropyl;

haloalkyl: an alkyl group as mentioned above in which some or all of the hydrogen atoms may be replaced by halogen atoms as mentioned above: in particular chloromethyl, bromomethyl, dichloromethyl, trichloromethyl, fluoromethyl, difluoromethyl, trifluoromethyl, chlorofluoromethyl, dichlorofluoromethyl, chlorodifluoromethyl;

alkoxyalkyl: a saturated straight-chain or mono-, di- or tribranched hydrocarbon chain which is interrupted by an oxygen atom, for example C₅-C₁₂-alkoxyalkyl: a hydrocarbon chain as described above having 5 to 12 carbon atoms which may be interrupted by an oxygen atom in any position, such as propoxyethyl, butoxyethyl, pentoxyethyl, hexyloxyethyl, heptyloxyethyl, octyloxyethyl, nonyloxyethyl, 3-(3-ethylhexyloxy)ethyl, 3-(2,4,4-trimethylpentyloxy)ethyl, 3-(1-ethyl-3-methylbutoxy)ethyl, ethoxypropyl, propoxypropyl, butoxypropyl, pentoxypropyl, hexyloxypropyl, heptyloxypropyl, octyloxy-propyl, nonyloxypropyl, 3-(3-ethylhexyloxy)propyl, 3-(2,4,4-trimethylpentyloxy)propyl, 3-(1-ethyl-3-methylbutoxy)propyl, ethoxybutyl, propoxybutyl, butoxybutyl, pentoxybutyl, hexyloxybutyl, heptyloxybutyl, octyloxybutyl, nonyloxybutyl, 3-(3-ethylhexyloxy)butyl, 3-(2,4,4-trimethylpentyloxy)butyl, 3-(1-ethyl-3-methylbutoxy)butyl, methoxypentyl, ethoxypentyl, propoxypentyl, butoxypentyl, pentoxypentyl, hexyloxypentyl, heptyl-oxypentyl, 3-(3-methylhexyloxy)pentyl, 3-(2,4-dimethylpentyloxy)pentyl, 3-(1-ethyl-3-methylbutoxy)pentyl;

alkynyl: straight-chain or branched hydrocarbon groups having 2 to 4, 6, 8 or 10 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;

cycloalkyl: mono- or bicyclic saturated hydrocarbon groups having 3 to 6 or 8 carbon ring members, for example C₃-C₈-cycloalkyl, such as cyclopropyl, cyclobutyl, cyclopentyl, cyclohexyl, cycloheptyl and cyclooctyl;

five- to ten-membered saturated, partially unsaturated or aromatic heterocycle comprising one to four heteroatoms from the group consisting of O, N and S:
5- or 6-membered heterocyclyl comprising one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 2-pyrrolin-2-yl, 2-pyrrolin-3-yl, 3-pyrrolin-2-yl, 3-pyrrolin-3-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl and 2-piperazinyl;
5-membered heteroaryl comprising one to four nitrogen atoms or one to three nitrogen atoms and one sulfur or oxygen atom: 5-membered heteroaryl groups which, in addition to carbon atoms, may comprise one to four nitrogen atoms or one to three nitrogen atoms and one sulfur or oxygen atom as ring members, for example 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl and 1,3,4-triazol-2-yl;
6-membered heteroaryl comprising one to three or one to four nitrogen atoms: 6-membered heteroaryl groups which, in addition to carbon atoms, may comprise one to three or one to four nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl;

alkylene: divalent unbranched or branched chains of 1 to 6 CH₂ groups which may carry up to four C₁-C₆-alkyl groups, for example CH₂, CH₂CH₂, CH(CH₃)CH₂, CH₂CH₂CH₂, CH₂CH₂CH₂CH₂, CH(CH₃)CH₂CH₂, CH(CH₂CH₃)CH₂CH₂ and CH₂CH₂CH₂CH₂CH₂;

alkenylene: divalent unbranched or branched chains of 2 to 6 CH₂ groups comprising one or more double bonds in any position, for example CH═CH, CH₂CH═CH, CH₂CH═CHCH₂, CH═CHCH═CH, CH═CHCH₂CH₂CH₂, CH₂CH═CHCH₂CH₂, CH₂C(CH₃)═CHCH₂CH₂ and C(CH₃)═CHCH₂CH₂CH₂;

alkynylene: divalent unbranched or branched chains of 2 to 6 CH₂ groups comprising one or more triple bonds in any position, for example C≡C, CH₂C═C, CH₂C═CCH₂, C≡CC≡C, C≡CCH₂CH₂CH₂, CH₂C≡CCH₂CH₂ and CH(CH₃)C≡CCH₂CH₂.

The scope of the present invention includes the (R)- and (S) isomers and the racemates of compounds of the formula I having chiral centers.

With a view to the intended use of the azolopyrimidines of the formula I, particular preference is given to the following meanings of the substituents, in each case on their own or in combination:

In one embodiment of the compounds I, the group Y is unbranched or is branched once; preferably, Y is unbranched C₁-C₆-alkylene, in particular C₁-C₄-alkylene. Particularly preferred meanings of Y are methylene and ethylene.

In a further embodiment of the compounds I, R¹ is not phenyl.

In a further embodiment of the compounds I there are one or two groups R¹ present, which preferably has the following meaning: halogen, C₁-C₆-alkyl and halomethyl.

In a further embodiment of the compounds I, R² is an ethyl or an n-propyl group.

In a further embodiment of the compounds I, R³ is hydrogen, NH₂ or C₁-C₆-alkyl, preferably hydrogen or NH₂, in particular hydrogen.

In a further embodiment of the compounds I, A is N or CH, in particular N.

In a further embodiment of the compounds I, A is C—R^{a}, where R^{a} is C₁-C₆-alkyl which may be substituted by one to three groups R^{b} according to claim 1.

In a further embodiment of the compounds I, the variables are as defined below:

Y is C₁-C₆-alkylene, optionally substituted by 1 to 4 C₁-C₆-alkyl groups;
R¹ is halogen, C₁-C₆-alkyl or halomethyl;
n is zero, 1, 2 or 3;
R² is C₂-C₆-alkyl, C₃-C₆-cycloalkyl or C₁-C₁₂-alkoxy-C₁-C₁₂-alkyl;
R³ is hydrogen, NH₂ or C₁-C₆-alkyl;

### A is N or C—R^{C},

R^{c} is hydrogen or C₁-C₆-alkyl.

In particular with a view to their use, preference is given to the compounds I compiled in the tables below. Moreover, the groups mentioned for a substituent in the tables are per se, independently of the combination in which they are mentioned, a particularly preferred embodiment of the substituent in question.

TABLE 1
Compounds of the formula I in which Y is CH₂, R² is ethyl, A is CH, R³
is hydrogen and R¹ₙ for each compound corresponds to one row of
Table A

TABLE 2
Compounds of the formula I in which Y is CH₂CH₂, R² is ethyl, A is CH,
R³ is hydrogen and R¹ₙ for each compound corresponds to one row of
Table A

TABLE 3
Compounds of the formula I in which Y is CH₂CH₂CH₂, R² is ethyl, A is
CH, R³ is hydrogen and R¹ₙ for each compound corresponds to one
row of Table A

TABLE 4
Compounds of the formula I in which Y is CH(CH₃)CH₂, R² is ethyl, A is
CH, R³ is hydrogen and R¹ₙ for each compound corresponds to
one row of Table A

TABLE 5
Compounds of the formula I in which Y is CH₂CH₂CH₂CH₂, R² is ethyl,
A is CH, R³ is hydrogen and R¹ₙ for each compound corresponds
to one row of Table A

TABLE 6
Compounds of the formula I in which Y is CH(CH₃)CH₂CH₂, R² is ethyl,
A is CH, R³ is hydrogen and R¹ₙ for each compound corresponds
to one row of Table A

TABLE 7
Compounds of the formula I in which Y is CH₂, R² is n-propyl, A is CH,
R³ is hydrogen and R¹ₙ for each compound corresponds to one row
of Table A

TABLE 8
Compounds of the formula I in which Y is CH₂CH₂, R² is n-propyl,
A is CH, R³ is hydrogen and R¹ₙ for each compound corresponds to one
row of Table A

TABLE 9
Compounds of the formula I in which Y is CH₂CH₂CH₂, R² is n-propyl,
A is CH, R³ is hydrogen and R¹ₙ for each compound corresponds to
one row of Table A

TABLE 10
Compounds of the formula I in which Y is CH(CH₃)CH₂, R² is n-propyl,
A is CH, R³ is hydrogen and R¹ₙ for each compound corresponds to
one row of Table A

TABLE 11

Compounds of the formula I in which Y is CH₂CH₂CH₂CH₂, R² is
n-propyl, A is CH, R³ is hydrogen and R¹ₙ for each compound
corresponds to one row of Table A

TABLE 12

Compounds of the formula I in which Y is CH(CH₃)CH₂CH₂, R² is
n-propyl, A is CH, R³ is hydrogen and R¹ₙ for each compound
corresponds to one row of Table A

TABLE 13
Compounds of the formula I in which Y is CH₂, R² is ethyl, A is N, R³ is
hydrogen and R¹ₙ for each compound corresponds to one row
of Table A

TABLE 14
Compounds of the formula I in which Y is CH₂CH₂, R² is ethyl, A is N,
R³ is hydrogen and R¹ₙ for each compound corresponds
to one row of Table A

TABLE 15

Compounds of the formula I in which Y is CH₂CH₂CH₂, R² is
ethyl, A is N, R³ is hydrogen and R¹ₙ for each compound
corresponds to one row of Table A

TABLE 16

Compounds of the formula I in which Y is CH(CH₃)CH₂, R² is
ethyl, A is N, R³ is hydrogen and R¹ₙ for each compound
corresponds to one row of Table A

TABLE 17

Compounds of the formula I in which Y is CH₂CH₂CH₂CH₂,
R² is ethyl, A is N, R³ is hydrogen and R¹ₙ for each
compound corresponds to one row of Table A

TABLE 18

Compounds of the formula I in which Y is CH(CH₃)CH₂CH₂,
R² is ethyl, A is N, R³ is hydrogen and R¹ₙ for each
compound corresponds to one row of Table A

TABLE 19

Compounds of the formula I in which Y is CH₂, R² is n-propyl,
A is N, R³ is hydrogen and R¹ₙ for each compound
corresponds to one row of Table A

TABLE 20

Compounds of the formula I in which Y is CH₂CH₂, R² is
n-propyl, A is N, R³ is hydrogen and R¹ₙ for each
compound corresponds to one row of Table A

TABLE 21

Compounds of the formula I in which Y is CH₂CH₂CH₂, R² is
n-propyl, A is N, R³ is hydrogen and R¹ₙ for each compound
corresponds to one row of Table A

TABLE 22

Compounds of the formula I in which Y is CH(CH₃)CH₂, R² is
n-propyl, A is N, R³ is hydrogen and R¹ₙ for each compound
corresponds to one row of Table A

TABLE 23

Compounds of the formula I in which Y is CH₂CH₂CH₂CH₂,
R² is n-propyl, A is N, R³ is hydrogen and R¹ₙ for each
compound corresponds to one row of Table A

TABLE 24

Compounds of the formula I in which Y is CH(CH₃)CH₂CH₂,
R² is n-propyl, A is N, R³ is hydrogen and R¹ₙ for each compound
corresponds to one row of Table A

TABLE 25
Compounds of the formula I in which Y is CH₂, R² is ethyl, A is CH, R³
is amino and R¹ₙ for each compound corresponds to one row of Table A

TABLE 26
Compounds of the formula I in which Y is CH₂CH₂, R² is ethyl, A is
CH, R³ is amino and R¹ₙ for each compound corresponds to one row of
Table A

TABLE 27

Compounds of the formula I in which Y is CH₂CH₂CH₂, R² is
ethyl, A is CH, R³ is amino and R¹ₙ for each compound
corresponds to one row of Table A

TABLE 28
Compounds of the formula I in which Y is CH(CH₃)CH₂, R² is ethyl, A is
CH, R³ is amino and R¹ₙ for each compound corresponds to one row of
Table A

TABLE 29
Compounds of the formula I in which Y is CH₂CH₂CH₂CH₂, R² is ethyl,
A is CH, R³ is amino and R¹ₙ for each compound corresponds
to one row of Table A

TABLE 30
Compounds of the formula I in which Y is CH(CH₃)CH₂CH₂, R² is ethyl,
A is CH, R³ is amino and R¹ₙ for each compound corresponds to one
row of Table A

TABLE 31
Compounds of the formula I in which Y is CH₂, R² is n-propyl, A is CH,
R³ is amino and R¹ₙ for each compound corresponds
to one row of Table A

TABLE 32
Compounds of the formula I in which Y is CH₂CH₂, R² is n-propyl, A is
CH, R³ is amino and R¹ₙ for each compound corresponds
to one row of Table A

TABLE 33
Compounds of the formula I in which Y is CH₂CH₂CH₂, R² is n-propyl,
A is CH, R³ is amino and R¹ₙ for each compound corresponds
to one row of Table A

TABLE 34
Compounds of the formula I in which Y is CH(CH₃)CH₂, R² is n-propyl,
A is CH, R³ is amino and R¹ₙ for each compound corresponds
to one row of Table A

TABLE 35

Compounds of the formula I in which Y is CH₂CH₂CH₂CH₂, R² is
n-propyl, A is CH, R^{3 is amino and R}¹ₙ for each compound
corresponds to one row of Table A

TABLE 36

Compounds of the formula I in which Y is CH(CH₃)CH₂CH₂, R² is
n-propyl, A is CH, R^{3 is amino and R}¹ₙ for each compound
corresponds to one row of Table A

TABLE 37
Compounds of the formula I in which Y is CH₂, R² is ethyl, A is N, R³ is
amino and R¹ₙ for each compound corresponds to one row of Table A

TABLE 38
Compounds of the formula I in which Y is CH₂CH₂, R² is ethyl, A is N,
R³ is amino and R¹ₙ for each compound corresponds
to one row of Table A

TABLE 39
Compounds of the formula I in which Y is CH₂CH₂CH₂, R² is ethyl,
A is N, R³ is amino and R¹ₙ for each compound corresponds
to one row of Table A

TABLE 40
Compounds of the formula I in which Y is CH(CH₃)CH₂, R² is ethyl,
A is N, R³ is amino and R¹ₙ for each compound corresponds
to one row of Table A

TABLE 41

Compounds of the formula I in which Y is CH₂CH₂CH₂CH₂, R² is
ethyl, A is N, R³ is amino and R¹ₙ for each compound corresponds
to one row of Table A

TABLE 42

Compounds of the formula I in which Y is CH(CH₃)CH₂CH₂, R² is
ethyl, A is N, R³ is amino and R¹ₙ for each compound corresponds
to one row of Table A

TABLE 43
Compounds of the formula I in which Y is CH₂, R² is n-propyl, A is N, R³
is amino and R¹ₙ for each compound corresponds to one row of Table A

TABLE 44

Compounds of the formula I in which Y is CH₂CH₂, R² is n-propyl,
A is N, R³ is amino and R¹ₙ for each compound corresponds to
one row of Table A

TABLE 45
Compounds of the formula I in which Y is CH₂CH₂CH₂, R² is n-propyl,
A is N, R³ is amino and R¹ₙ for each compound corresponds to one
row of Table A

TABLE 46
Compounds of the formula I in which Y is CH(CH₃)CH₂, R² is n-propyl,
A is N, R³ is amino and R¹ₙ for each compound corresponds to one
row of Table A

TABLE 47
Compounds of the formula I in which Y is CH₂CH₂CH₂CH₂, R² is
n-propyl, A is N, R³ is amino and R¹ₙ for each compound corresponds to
one row of Table A

TABLE 48
Compounds of the formula I in which Y is CH(CH₃)CH₂CH₂, R² is
n-propyl, A is N, R³ is amino and R¹ₙ for each compound corresponds to
one row of Table A

TABLE 49

Compounds of the formula I in which Y is CH₂, R² is ethyl, A is CH,
R³ is methyl and R¹ₙ for each compound corresponds to
one row of Table A

TABLE 50
Compounds of the formula I in which Y is CH₂CH₂, R² is ethyl, A is CH,
R³ is methyl and R¹ₙ for each compound corresponds to
one row of Table A

TABLE 51

Compounds of the formula I in which Y is CH₂CH₂CH₂, R² is ethyl,
A is CH, R³ is methyl and R¹ₙ for each compound corresponds to
one row of Table A

TABLE 52

Compounds of the formula I in which Y is CH(CH₃)CH₂, R² is ethyl,
A is CH, R³ is methyl and R¹ₙ for each compound corresponds
to one row of Table A

TABLE 53

Compounds of the formula I in which Y is CH₂CH₂CH₂CH₂,
R² is ethyl, A is CH, R³ is methyl and R¹ₙ for each
compound corresponds to one row of Table A

TABLE 54
Compounds of the formula I in which Y is CH(CH₃)CH₂CH₂, R² is ethyl,
A is CH, R³ is methyl and R¹ₙ for each compound corresponds to
one row of Table A

TABLE 55
Compounds of the formula I in which Y is CH₂, R² is n-propyl, A is CH,
R³ is methyl and R¹ₙ for each compound corresponds to
one row of Table A

TABLE 56

Compounds of the formula I in which Y is CH₂CH₂, R² is
n-propyl, A is CH, R³ is methyl and R¹ₙ for each compound
corresponds to one row of Table A

TABLE 57

Compounds of the formula I in which Y is CH₂CH₂CH₂, R² is
n-propyl, A is CH, R³ is methyl and R¹ₙ for each compound
corresponds to one row of Table A

TABLE 58
Compounds of the formula I in which Y is CH(CH₃)CH₂, R² is n-propyl,
A is CH, R³ is methyl and R¹ₙ for each compound corresponds
to one row of Table A

TABLE 59

Compounds of the formula I in which Y is CH₂CH₂CH₂CH₂,
R² is n-propyl, A is CH, R³ is methyl and R¹ₙ for each compound
corresponds to one row of Table A

TABLE 60

Compounds of the formula I in which Y is CH(CH₃)CH₂CH₂,
R² is n-propyl, A is CH, R³ is methyl and R¹ₙ for each
compound corresponds to one row of Table A

TABLE 61

Compounds of the formula I in which Y is CH₂, R² is ethyl,
A is N, R³ is methyl and R¹ₙ for each compound
corresponds to one row of Table A

TABLE 62

Compounds of the formula I in which Y is CH₂CH₂, R² is ethyl,
A is N, R³ is methyl and R¹ₙ for each compound corresponds to one
row of Table A

TABLE 63

Compounds of the formula I in which Y is CH₂CH₂CH₂, R² is
ethyl, A is N, R³ is methyl and R¹ₙ for each compound corresponds
to one row of Table A

TABLE 64

Compounds of the formula I in which Y is CH(CH₃)CH₂, R² is ethyl,
A is N, R³ is methyl and R¹ₙ for each compound corresponds to one
row of Table A

TABLE 65

Compounds of the formula I in which Y is CH₂CH₂CH₂CH₂,
R² is ethyl, A is N, R³ is methyl and R¹ₙ for each compound
corresponds to one row of Table A

TABLE 66

Compounds of the formula I in which Y is CH(CH₃)CH₂CH₂,
R² is ethyl, A is N, R³ is methyl and R¹ₙ for each compound
corresponds to one row of Table A

TABLE 67
Compounds of the formula I in which Y is CH₂, R² is n-propyl, A is N,
R³ is methyl and R¹ₙ for each compound corresponds to one row of
Table A

TABLE 68
Compounds of the formula I in which Y is CH₂CH₂, R² is n-propyl, A is
N, R³ is methyl and R¹ₙ for each compound corresponds to one
row of Table A

TABLE 69
Compounds of the formula I in which Y is CH₂CH₂CH₂, R² is
n-propyl, A is N, R³ is methyl and R¹ₙ for each compound corresponds
to one row of Table A

TABLE 70
Compounds of the formula I in which Y is CH(CH₃)CH₂, R² is n-propyl,
A is N, R³ is methyl and R¹ₙ for each compound corresponds to one
row of Table A

TABLE 71

Compounds of the formula I in which Y is CH₂CH₂CH₂CH₂,
R² is n-propyl, A is N, R³ is methyl and R¹ₙ for each compound
corresponds to one row of Table A

TABLE 72

Compounds of the formula I in which Y is CH(CH₃)CH₂CH₂,
R² is n-propyl, A is N, R³ is methyl and R¹ₙ for each
compound corresponds to one row of Table A

TABLE A

| No. | R¹ₙ |
|---|---|
| A-1 | — |
| A-2 | 2-CH₃ |
| A-3 | 3-CH₃ |
| A-4 | 4-CH₃ |
| A-5 | 2-CH₂CH₃ |
| A-6 | 3-CH₂CH₃ |
| A-7 | 4-CH₂CH₃ |
| A-8 | 2-CH(CH₃)₂ |
| A-9 | 3-CH(CH₃)₂ |
| A-10 | 4-CH(CH₃)₂ |
| A-11 | 2-CH₂CH₂CH₃ |
| A-12 | 3-CH₂CH₂CH₃ |
| A-13 | 4-CH₂CH₂CH₃ |
| A-14 | 2-C(CH₃)₃ |
| A-15 | 3-C(CH₃)₃ |
| A-16 | 4-C(CH₃)₃ |
| A-17 | 2-CH(CH₃)CH₂CH₃ |
| A-18 | 3-CH(CH₃)CH₂CH₃ |
| A-19 | 4-CH(CH₃)CH₂CH₃ |
| A-20 | 2-CF₃ |
| A-21 | 3-CF₃ |
| A-22 | 4-CF₃ |
| A-23 | 2-Cl |
| A-24 | 3-Cl |
| A-25 | 4-Cl |
| A-26 | 2-F |
| A-27 | 3-F |
| A-28 | 4-F |
| A-29 | 2-Br |
| A-30 | 3-Br |
| A-31 | 4-Br |
| A-32 | 2,3-(CH₃)₂ |
| A-33 | 2,4-(CH₃)₂ |
| A-34 | 2,5-(CH₃)₂ |
| A-35 | 2,6-(CH₃)₂ |
| A-36 | 3,4-(CH₃)₂ |
| A-37 | 3,5-(CH₃)₂ |
| A-38 | 2,3-(CH₂CH₃)₂ |
| A-39 | 2,4-(CH₂CH₃)₂ |
| A-40 | 2,5-(CH₂CH₃)₂ |
| A-41 | 2,6-(CH₂CH₃)₂ |
| A-42 | 3,4-(CH₂CH₃)₂ |
| A-43 | 3,5-(CH₂CH₃)₂ |
| A-44 | 2,3-[CH(CH₃)₂]₂ |
| A-45 | 2,4-[CH(CH₃)₂]₂ |
| A-46 | 2,5-[CH(CH₃)₂]₂ |
| A-47 | 2,6-[CH(CH₃)₂]₂ |
| A-48 | 3,4-[CH(CH₃)₂]₂ |
| A-49 | 3,5-[CH(CH₃)₂]₂ |
| A-50 | 2,3-(CH₂CH₂CH₃)₂ |
| A-51 | 2,4-(CH₂CH₂CH₃)₂ |
| A-52 | 2,5-(CH₂CH₂CH₃)₂ |
| A-53 | 2,6-(CH₂CH₂CH₃)₂ |
| A-54 | 3,4-(CH₂CH₂CH₃)₂ |
| A-55 | 3,5-(CH₂CH₂CH₃)₂ |
| A-56 | 2,3-(CF₃)₂ |
| A-57 | 2,4-(CF₃)₂ |
| A-58 | 2,5-(CF₃)₂ |
| A-59 | 2,6-(CF₃)₂ |
| A-60 | 3,4-(CF₃)₂ |
| A-61 | 3,5-(CF₃)₂ |
| A-62 | 2,3-Cl₂ |
| A-63 | 2,4-Cl₂ |
| A-64 | 2,5-Cl₂ |
| A-65 | 2,6-Cl₂ |
| A-66 | 3,4-Cl₂ |
| A-67 | 3,5-Cl₂ |
| A-68 | 2,3-F₂ |
| A-69 | 2,4-F₂ |
| A-70 | 2,5-F₂ |
| A-71 | 2,6-F₂ |
| A-72 | 3,4-F₂ |
| A-73 | 3,5-F₂ |
| A-74 | 2-Br |
| A-75 | 3-Br |
| A-76 | 4-Br |
| A-77 | 2,3-Br₂ |
| A-78 | 2,4-Br₂ |
| A-79 | 2,5-Br₂ |
| A-80 | 2,6-Br₂ |
| A-81 | 3,4-Br₂ |
| A-82 | 3,5-Br₂ |
| A-83 | 2,3,4-(CH₃)₃ |
| A-84 | 2,3,5-(CH₃)₃ |
| A-85 | 2,3,6-(CH₃)₃ |
| A-86 | 2,4,5-(CH₃)₃ |
| A-87 | 2,4,6-(CH₃)₃ |
| A-88 | 2,6-(CH₃)₂ |
| A-89 | 3,4-(CH₃)₂ |
| A-90 | 3,5-(CH₃)₂ |
| A-91 | 2,3,4-Cl₃ |
| A-92 | 2,3,5-Cl₃ |
| A-93 | 2,3,6-Cl₃ |
| A-94 | 2,4,5-Cl₃ |
| A-95 | 2,4,6-Cl₃ |
| A-96 | 2,3,4-F₃ |
| A-97 | 2,3,5-F₃ |
| A-98 | 2,3,6-F₃ |
| A-99 | 2,4,5-F₃ |
| A-100 | 2,4,6-F₃ |
| A-101 | 2,3,4-Br₃ |
| A-102 | 2,3,5-Br₃ |
| A-103 | 2,3,6-Br₃ |
| A-104 | 2,4,5-Br₃ |
| A-105 | 2,4,6-Br₃ |

The compounds I are suitable as fungicides. They are distinguished by an outstanding effectiveness against a broad spectrum of phytopathogenic fungi from the class of the Ascomycetes, Deuteromycetes, Oomycetes and Basidiomycetes, especially from the class of the Oomycetes. Some are systemically effective and they can be used in plant protection as foliar fungicides, as fungicides for seed dressing and as soil fungicides.

They are particularly important in the control of a multitude of fungi on various cultivated plants, such as wheat, rye, barley, oats, rice, corn, grass, bananas, cotton, soybeans, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:
Alternaria species on vegetables, oilseed rape, sugar beet and fruit and rice, such as, for example, A. solani or A. alternata on potatoes and tomatoes;
Aphanomyces species on sugar beet and vegetables;
Ascochyta species on cereals and vegetables;
Bipolaris and Drechslera species on corn, cereals, rice and lawns, such as, for example, D. maydis on corn;
Blumeria graminis (powdery mildew) on cereals;
Botrytis cinerea (gray mold) on strawberries, vegetables, flowers and grapevines;
Bremia Iactucae on lettuce;
Cercospora species on corn, soybeans, rice and sugar beet;
Cochliobolus species on corn, cereals, rice, such as, for example Cochliobolus sativus on cereals, Cochiobolus miyabeanus on rice;
Colletotricum species on soybeans and cotton;
Drechslera species, Pyrenophora species on corn, cereals, rice and lawns, such as, for example, D. teres on barley or D. tritici-repentis on wheat;
Esca on grapevines, caused by Phaeoacremonium chlamydosporium, Ph. Aleophilum and Formitipora punctata (syn. Phellinus punctatus);
Exserohilum species on corn;
Erysiphe cichoracearum and Sphaerotheca fuliginea on cucumbers;
Fusarium and Verticillium species on various plants, such as, for example, F. graminearum or F. culmorum on cereals or F. oxysporum on a multitude of plants, such as, for example, tomatoes;
Gaeumanomyces graminis on cereals;
Gibberella species on cereals and rice (for example Gibberella fujikuroi on rice);
Grainstaining complex on rice;
Helminthosporium species on corn and rice;
Michrodochium nivale on cereals;
Mycosphaerella species on cereals, bananas and groundnuts, such as, for example, M. graminicola on wheat or M. fijiensis on bananas;
Peronospora species on cabbage and bulbous plants, such as, for example, P. brassicae on cabbage or P. destructor on onions;
Phakopsara pachyrhizi and Phakopsara meibomiae on soybeans;
Phomopsis species on soybeans and sunflowers;
Phytophthora infestans on potatoes and tomatoes;
Phytophthora species on various plants, such as, for example, P. capsici on bell pepper;
Plasmopara viticola on grapevines;
Podosphaera leucotricha on apples;
Pseudocercosporella herpotrichoides on cereals;
Pseudoperonospora on various plants, such as, for example, P. cubensis on cucumber or P. humili on hops;
Puccinia species on various plants, such as, for example, P. triticina, P. striformins, P. hordei or P. graminis on cereals or P. asparagi on asparagus;
Pyricularia oryzae, Corticium sasakii, Sarocladium oryzae, S. attenuatum, Entyloma oryzae on rice;
Pyricularia grisea on lawns and cereals;
Pythium spp. on lawns, rice, corn, cotton, oilseed rape, sunflowers, sugar beet, vegetables and other plants, such as, for example, P. ultiumum on various plants, P. aphanidermatum on lawns;
Rhizoctonia species on cotton, rice, potatoes, lawns, corn, oilseed rape, potatoes, sugar beet, vegetables and on various plants, such as, for example, R. solani on beet and various plants;
Rhynchosporium secalis on barley, rye and triticale;
Sclerotinia species on oilseed rape and sunflowers;
Septoria tritici and Stagonospora nodorum on wheat;
Erysiphe (syn. Uncinula) necator on grapevines;
Setospaeria species on corn and lawns;
Sphacelotheca reilinia on corn;
Thievaliopsis species on soybeans and cotton;
Tilletia species on cereals;
Ustilago species on cereals, corn and sugar cane, such as, for example, U. maydis on corn;
Venturia species (scab) on apples and pears, such as, for example, V. inaequalis on apples.

They are particularly suitable for controlling harmful fungi from the class of the Oomycetes, such as Peronospora species, Phytophthora species, Plasmopara viticola and Pseudoperonospora species.

The compounds I are also suitable for controlling harmful fungi in the protection of materials (for example wood, paper, paint dispersions, fibers or fabrics) and in the protection of stored products. In the protection of wood, particular attention is paid to the following harmful fungi:

Ascomycetes, such as Ophiostoma spp., Ceratocystis spp., Aureobasidium pullulans, Sclerophoma spp., Chaetomium spp., Humicola spp., Petriella spp., Trichurus spp.; Basidiomycetes, such as Coniophora spp., Coriolus spp., Gloeophyllum spp., Lentinus spp., Pleurotus spp., Poria spp., Serpula spp. and Tyromyces spp., Deuteromycetes, such as Aspergillus spp., Cladosporium spp., Penicillium spp., Trichoderma spp., Alternaria spp., Paecilomyces spp. and Zygomycetes, such as Mucor spp., additionally in the protection of materials the following yeasts: Candida spp. and Saccharomyces cerevisae.

The compounds I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise from 0.1 to 95%, preferably from 0.5 to 90%, by weight of active compound.

When employed in plant protection, the amounts applied are, depending on the kind of effect desired, from 0.01 to 2.0 kg of active compound per ha. In seed treatment, amounts of active compound of 1 to 1000 g/100 kg, preferably 5 to 100 g/100 kg, of seed are generally required.

When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound per cubic meter of treated material.

The compounds of the formula I can be present in various crystal modifications which may differ in their biological activity. They also form part of the subject matter of the present invention.

The compounds I can be converted into the customary formulations, for example solutions, emulsions, suspensions, dusts, powders, pastes and granules. The application form depends on the particular purpose; in each case, it should ensure a fine and uniform distribution of the compound according to the invention.

The formulations are prepared in a known manner, for example by extending the active compound with solvents and/or carriers, if desired using emulsifiers and dispersants. Solvents/auxiliaries which are suitable are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example highly disperse silica, silicates); emulsifiers such as nonionic and anionic emulsifiers (for example polyoxyethylene fatty alcohol ethers, alkylsulfonates and arylsulfonates) and dispersants such as lignosulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ethers, ethoxylated isooctylphenol, octylphenol, nonylphenol, alkylphenol polyglycol ethers, tributylphenyl polyglycol ethers, tristearylphenyl polyglycol ethers, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

The following are examples of formulations: 1. Products for dilution with water

### A Water-Soluble Concentrates (SL, LS)

10 parts by weight of the active compounds are dissolved in 90 parts by weight of water or in a water-soluble solvent. As an alternative, wetting agents or other auxiliaries are added. The active compound dissolves upon dilution with water. In this way, a formulation having a content of 10% by weight of active compound is obtained.

### B Dispersible Concentrates (DC)

20 parts by weight of the active compounds are dissolved in 70 parts by weight of cyclohexanone with addition of 10 parts by weight of a dispersant, for example polyvinylpyrrolidone. Dilution with water gives a dispersion. The active compound content is 20% by weight

### C Emulsifiable Concentrates (EC)

15 parts by weight of the active compounds are dissolved in 75 parts by weight of xylene with addition of calcium dodecylbenzenesulfonate and castor oil ethoxylate (in each case 5 parts by weight). Dilution with water gives an emulsion. The formulation has an active compound content of 15% by weight.

D Emulsions (EW, EO, ES)

25 parts by weight of the active compounds are dissolved in 35 parts by weight of xylene with addition of calcium dodecylbenzenesulfonate and castor oil ethoxylate (in each case 5 parts by weight). This mixture is introduced into 30 parts by weight of water by means of an emulsifying machine (e.g. Ultraturrax) and made into a homogeneous emulsion. Dilution with water gives an emulsion. The formulation has an active compound content of 25% by weight.

### E Suspensions (SC, OD, FS)

In an agitated ball mill, 20 parts by weight of the active compounds are comminuted with addition of 10 parts by weight of dispersants and wetting agents and 70 parts by weight of water or an organic solvent to give a fine active compound suspension. Dilution with water gives a stable suspension of the active compound. The active compound content in the formulation is 20% by weight.

F Water-Dispersible Granules and Water-Soluble Granules (WG, SG)

50 parts by weight of the active compounds are ground finely with addition of 50 parts by weight of dispersants and wetting agents and prepared as water-dispersible or water-soluble granules by means of technical appliances (for example extrusion, spray tower, fluidized bed). Dilution with water gives a stable dispersion or solution of the active compound. The formulation has an active compound content of 50% by weight.

### G Water-Dispersible Powders and Water-Soluble Powders (WP, SP, SS, WS)

75 parts by weight of the active compounds are ground in a rotor-stator mill with addition of 25 parts by weight of dispersants, wetting agents and silica gel. Dilution with water gives a stable dispersion or solution of the active compound. The active compound content of the formulation is 75% by weight.

### H Gel Formulations

In a ball mill, 20 parts by weight of the active compounds, 10 parts by weight of dispersant, 1 part by weight of gelling agent and 70 parts by weight of water or an organic solvent are ground to give a fine suspension. On dilution with water, a stable suspension having an active compound content of 20% by weight is obtained.

### 2. Products to be Applied Undiluted

### I Dustable Powders (DP, DS)

5 parts by weight of the active compounds are ground finely and mixed intimately with 95 parts by weight of finely divided kaolin. This gives a dustable product having an active compound content of 5% by weight.

### J Granules (GR, FG, GG, MG)

0.5 part by weight of the active compounds is ground finely and associated with 99.5 parts by weight of carriers. Current methods are extrusion, spray-drying or the fluidized bed. This gives granules to be applied undiluted having an active compound content of 0.5% by weight.

### K ULV Solutions (UL)

10 parts by weight of the active compounds are dissolved in 90 parts by weight of an organic solvent, for example xylene. This gives a product to be applied undiluted having an active compound content of 10% by weight.

For seed treatment, use is usually made of water-soluble concentrates (LS), suspensions (FS), dustable powders (DS), water-dispersible and water-soluble powders (WS, SS), emulsions (ES), emulsifiable concentrates (EC) and gel formulations (GF). These formulations can be applied to the seed in undiluted form or, preferably, diluted. Application can be carried out prior to sowing.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; the intention is to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply formulations comprising over 95% by weight of active compound, or even to apply the active compound without additives.

Various types of oils, wetters, adjuvants, herbicides, fungicides, other pesticides, or bactericides may be added to the active compounds, if appropriate not until immediately prior to use (tank mix). These agents can be admixed with the agents according to the invention in a weight ratio of 1:100 to 100:1, preferably 1:10 to 10:1.

Suitable adjuvants in this sense are in particular: organically modified polysiloxanes, for example Break Thru S 240®; alcohol alkoxylates, for example Atplus 245®, Atplus MBA 1303®, Plurafac LF 300® and Lutensol ON 30®; EO/PO block polymers, for example Pluronic RPE 2035® and Genapol B®; alcohol ethoxylates, for example Lutensol XP 80®; and sodium dioctylsulfosuccinate, for example Leophen R^{A}®.

The compositions according to the invention can, in the use form as fungicides, also be present together with other active compounds, e.g. with herbicides, insecticides, growth regulators, fungicides or else with fertilizers. Mixing the compounds I or the compositions comprising them in the application form as fungicides with other active compounds, in particular fungicides, it is in many cases possible to broaden the activity spectrum or to prevent the development of resistance. In many cases, synergistic effects are obtained.

The following list of fungicides, in conjunction with which the compounds according to the invention can be used, is intended to illustrate the possible combinations but does not limit them:

### Strobilurins

azoxystrobin, dimoxystrobin, enestroburin, fluoxastrobin, kresoxim-methyl, metominostrobin, picoxystrobin, pyraclostrobin, trifloxystrobin, orysastrobin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate, methyl 2-(ortho-(2,5-dimethylphenyloxymethylene)phenyl)-3-methoxyacrylate;

### Carboxamides

carboxanilides: benalaxyl, benodanil, boscalid, carboxin, mepronil, fenfuram, fenhexamid, flutolanil, furametpyr, metalaxyl, ofurace, oxadixyl, oxycarboxin, penthiopyrad, thifluzamide, tiadinil, N-(4′-bromobiphenyl-2-yl)-4-difluoromethyl-2-methylthiazole-5-carboxamide, N-(4′-trifluoromethylbiphenyl-2-yl)-4-difluoromethyl-2-methylthiazole-5-carboxamide, N-(4′-chloro-3′-fluorobiphenyl-2-yl)-4-difluoro-methyl-2-methylthiazole-5-carboxamide, N-(3′,4′-dichloro-4-fluorobiphenyl-2-yl)-3-difluoromethyl-1-methylpyrazole-4-carboxamide, N-(2-cyanophenyl)-3,4-dichloro-isothiazole-5-carboxamide;
carboxylic acid morpholides: dimethomorph, flumorph;
benzamides: flumetover, fluopicolide (picobenzamid), zoxamide;
other carboxamides: carpropamid, diclocymet, mandipropamid, N-(2-(4-[3-(4-chloro-phenyl)prop-2-ynyloxy]-3-methoxyphenyl)ethyl)-2-methanesulfonylamino-3-methyl-butyramide, N-(2-(4-[3-(4-chlorophenyl)prop-2-ynyloxy]-3-methoxyphenyl)ethyl)-2-ethanesulfonylamino-3-methylbutyramide;

### Azoles

triazoles: bitertanol, bromuconazole, cyproconazole, dienoconazole, diniconazole, enilconazole, epoxiconazole, fenbuconazole, flusilazole, fluquinconazole, flutriafol, hexaconazole, imibenconazole, ipconazole, metconazole, myclobutanil, penconazole, propiconazole, prothioconazole, simeconazole, tebuconazole, tetraconazole, triadimenol, triadimefon, triticonazole;
imidazoles: cyazofamid, imazalil, pefurazoate, prochloraz, triflumizole;
benzimidazoles: benomyl, carbendazim, fuberidazole, thiabendazole;
others: ethaboxam, etridiazole, hymexazole;

### Nitrogenous Heterocyclyl Compounds

pyridines: fluazinam, pyrifenox, 3-[5-(4-chlorophenyl)-2,3-dimethylisoxazolidin-3-yl]-pyridine;
pyrimidines: bupirimate, cyprodinil, ferimzone, fenarimol, mepanipyrim, nuarimol,
pyrimethanil;
piperazines: triforine:
pyrroles: fludioxonil, fenpicionil;
morpholines: aldimorph, dodemorph, fenpropimorph, tridemorph;
dicarboximides: iprodione, procymidone, vinclozolin;
others: acibenzolar-5-methyl, anilazine, captan, captafol, dazomet, diclomezine, fenoxanil, folpet, fenpropidin, famoxadone, fenamidone, octhilinone, probenazole, proquinazid, pyroquilon, quinoxyfen, tricyclazole, 5-chloro-7-(4-methylpiperidin-1-yl)-6-(2,4,6-trifluorophenyl)-[1,2,4]triazolo[1,5-a]pyrimidine, 2-butoxy-6-iodo-3-propyl-chromen-4-one, N,N-dimethyl-3-(3-bromo-6-fluoro-2-methylindole-1-sulfonyl)-[1,2,4]triazole-1-sulfonamide;

### Carbamates and Dithiocarbamates

dithiocarbamates: ferbam, mancozeb, maneb, metiram, metam, propineb, thiram, zineb, ziram;
carbamates: diethofencarb, flubenthiavalicarb, iprovalicarb, propamocarb, methyl 3-(4-chlorophenyl)-3-(2-isopropoxycarbonylamino-3-methylbutyrylamino)propionate, 4-fluorophenyl N-(1-(1-(4-cyanophenyl)ethanesulfonyl)but-2-yl)carbamate;

### Other Fungicides

guanidines: dodine, iminoctadine, guazatine;
antibiotics: kasugamycin, polyoxins, streptomycin, validamycin A;
organometailic compounds: fentin salts;
sulfur-containing heterocyclyl compounds: isoprothiolane, dithianon;
organophosphorus compounds: edifenphos, fosetyl, fosetyl-aluminum, iprobenfos, pyrazophos, tolclofos-methyl, phosphorous acid and its salts;
organochlorine compounds: thiophanate-methyl, chlorothalonil, dichlofluanid, tolylfluanid, flusulfamide, phthalide, hexachlorobenzene, pencycuron, quintozene;
nitrophenyl derivatives: binapacryl, dinocap, dinobuton;
inorganic active compounds: Bordeaux mixture, copper acetate, copper hydroxide, copper oxychloride, basic copper sulfate, sulfur;
others: spiroxamine, cyflufenamid, cymoxanil, metrafenone.

### SYNTHESIS EXAMPLES

The procedures described in the synthesis examples below were used to prepare further compounds I by appropriate modification of the starting materials. The compounds thus obtained are listed in the tables below, together with physical data.

### Example 1

### Preparation of 4-cyano-6-phenylhexan-3-one

With stirring and cooling with ice, 150 ml of a 1.6 M solution of n-butyllithium in n-hexane were added dropwise to a solution of 24.24 g of diisopropylamine in 80 ml of anhydrous THF. After 10 minutes, the mixture was cooled to −70° C. and a solution of 17.4 g of 4-phenylbutyronitrile in 20 ml of THF and a solution of 17.7 g of ethyl propionate in 20 ml of THF were successively added dropwise. Over a period of one hour, the temperature was then allowed to rise to 0° C. and the mixture was hydrolyzed with 150 ml of water. The organic phase was separated off and discarded and the aqueous phase was washed with 100 ml of cyclohexane, acidified to pH=2 with dil. hydrochloric acid and extracted with methyl tert-butyl ether (MTBE). The combined organic phases were washed with water and dried, and the solvent was removed. This gave, as a pale yellow oil, 20.6 g of the title compound as a 98% pure product (GC). ¹H-NMR: δ=0.92 (t); 1.07 (t); 1.97-2.25 (m); 2.3-2.4 (m); 2.6-2.78 (m), 4.06 (dd); 7.15-7.35 (m); 10.4 (s).

### Example 2

### Preparation of 7-amino-5-ethyl-6-(2-phenylethyl)-(1,2,4)-triazolo-(1,5-a)-pyrimidine [I-1]

8.57 g of 3-amino-1,2,4-triazole, 20.5 g of 4-cyano-6-phenylhexan-3-one and 3.8 g of p-toluenesulfonic acid in 80 ml of mesitylene were heated at 180° C. for 3.5 hours with continuous distillative removal of solvent. The remaining mesitylene was distilled off under reduced pressure and the residue was crystallized by digestion with water/MTBE. This solid was filtered off with suction and washed with water and MTBE. This gave 19.4 g of the title compound of m.p. 210-211° C.

### Example 3

### Preparation of ethyl 3-oxo-2-(2-phenylethyl)hexanoate

10 g of ethyl 3-oxohexanoate were added to 22.6 g of a 20% strength ethanolic solution of sodium ethoxide. After brief stirring, 11.7 g of 2-phenylethyl bromide were added dropwise and the solution was heated under reflux for 20 hours. The solvent was then distilled off and the residue was digested with 50 ml of water and 50 ml of methyl tert-butyl ether (MTBE). After phase separation, the aqueous was extracted with MTBE. The combined organic phases were washed with water and then dried, and the solvent was removed. This gave 13.4 g of an oil which was reacted further without further purification. Purity according to GC: 68%.

### Example 4

### Preparation of 7-hydroxy-6-(2-phenylethyl)-5-propyl-(1,2,4)-triazolo-(1,5-a)-pyrimidine

A solution of 1.3 g of 3-amino-1H-1,2,4-triazole, 6.1 g of the ester from Ex. 3 and 0.59 g of p-toluenesulfonic acid in 25 ml of mesitylene was stirred at 170° C. for 3.5 hours, during which time some solvent distilled off. The solvent was then distilled off under reduced pressure and the residue was taken up in dichloromethane. The dichloromethane phase was washed with saturated NaHCO₃ solution and water and then dried, and the solvent was removed. This gave 1.35 g of the title compound. ¹H-NMR (DMSO-d₆): δ=0.90 (t, 3H); 1.51 (2H); 2.43 (t, 3H); 2.74 (s, 4H); 7.14-7.24 (m, 3H); 7.29 (t, 2H); 8.19 (s, 1H); 12.94 (s, 1H).

### Example 5

### Preparation of 7-chloro-6-(2-phenylethyl)-5-propyl-(1,2,4)-triazolo-(1,5-a)-pyrimidine

1.35 g of the hydroxytriazolopyrimidine from Ex. 4 in 10 ml of phosphorus oxychloride were heated under reflux for four hours. Excess phosphorus oxychloride was then distilled off and the residue was taken up in dichloromethane. This solution was stirred into water. The organic phase was separated off, washed with saturated NaHCO₃ solution and water and then dried, and the solvent was removed. This gave 1.3 g of the title compound as an oil.

¹H-NMR (CDCl₃): δ=1.05 (t, 3H); 1.89 (2H); 2.86-2.92 (m, 4H); 3.18 (t, 2H); 7.20 (d, 2H); 7.28 (t, 1H); 7.33 (t, 2H); 8.47 (s, 1H).

### Example 6

### Preparation of 7-amino-6-(2-phenylethyl)-5-propyl-(1,2,4)-triazolo-(1,5-a)-pyrimidine [I-2]

In an autoclave, a solution of 1.3 g of the chlorotriazolopyrimidine from Ex. 5 and 1.1 ml of liquid ammonia in 50 ml anhydrous 1,4-dioxane was stirred at 130° C. under intrinsic pressure. The solvent was then distilled off and the residue was digested with dichloromethane/water. The organic phase was separated off, washed with water and dried, and the solvent was removed. Trituration with MTBE gave 0.52 g of the title compound of m.p. 190-191° C.

TABLE I
Compounds of the formula I

| | | | | | | Phys. data |
|---|---|---|---|---|---|---|
| No. | Y | R¹ₙ | R² | R³ | A | (m.p. [° C.]) |
| I-1 | CH₂CH₂ | — | CH₂CH₃ | H | N | 210-211 |
| I-2 | CH₂CH₂ | — | CH₂CH₂CH₃ | H | N | 190-191 |
| I-3 | CH₂CH₂CH₂ | — | CH₂CH₃ | H | N | 236-238 |
| I-4 | CH₂CH₂CH₂ | — | CH₂CH₃ | CH₃ | N | 198-200 |
| I-5 | CH₂CH₂CH₂ | — | CH₂CH₂CH₃ | H | N | 179-180 |
| I-6 | CH₂CH₂CH₂CH₂ | — | CH₂CH₃ | H | N | 178-180 |
| I-7 | CH₂CH₂CH₂C(CH₃)₂ | — | CH₂CH₂CH₃ | H | N | 204-206 |
| I-8 | CH₂CH₂CH₂C(CH₃)₂ | — | CH₂CH₂CH₃ | CH₃ | N | 180-181 |
| I-9 | CH₂CH₂CH₂CH₂CH₂ | — | CH₂CH₃ | H | N | 204-205 |
| I-10 | CH₂ | 4-Cl | CH₂CH₃ | H | N | 239-241 |
| I-11 | CH₂ | 3-CH₃ | CH₂CH₃ | H | N | 207-209 |
| I-12 | CH₂ | 3-CF₃ | CH₂CH₃ | H | N | 224-226 |
| I-13 | CH₂ | 2-CH₃ | CH₂CH₃ | H | N | 254-256 |
| I-14 | CH₂ | 4-CH₃ | CH₂CH₃ | H | N | 220-222 |
| I-15 | CH₂ | 4-C(CH₃)₃ | CH₂CH₃ | H | N | 236-238 |
| I-16 | CH₂ | 4-CF₃ | CH₂CH₃ | H | N | 213-215 |
| I-17 | CH₂ | 3,5-(CH₃)₂ | CH₂CH₃ | H | N | 242-244 |

Examples of the Action Against Harmful Fungi

The fungicidal action of the compounds of the formula I was demonstrated by the following experiments:

The active compounds were prepared as a stock solution comprising 25 mg of active compound which was made up to 10 ml using a mixture of acetone and/or DMSO and the emulsifier Uniperol® EL (wetting agent having emulsifying and dispersing action based on ethoxylated alkylphenols) in a volume ratio of solvent/emulsifier of 99/1. The mixture was then made up to 100 ml with water. This stock solution was diluted with the solvent/emulsifier/water mixture described to the concentration of active compounds stated below.

Compound A, known as Example No. 9 from EP-A 141 317, was used as comparative active compound:

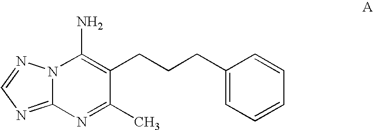

### Comparative Example 1

### Activity Against peronospora of Grapevines Caused by Plasmopara viticola

Leaves of potted vines were sprayed to runoff point with an aqueous suspension having the concentration of active compounds stated below. The next day, the undersides of the leaves were inoculated with an aqueous sporangia suspension of Plasmopara viticola. The vines were then initially placed in a water-vapor-saturated chamber at 24° C. for 48 hours and then in a greenhouse at temperatures between 20° C. and 30° C. for 5 days. After this time, the plants were once more placed in a humid chamber for 16 hours to accelerate the eruption of sporangiospores. The extent of the development of the infection on the undersides of the leaves was then determined visually.

In this test, the plants which had been treated with 250 ppm of the active compound I-5 showed an infection of 5%, whereas the plants which had been treated with 250 ppm of the comparative active compound A were 90% infected, the plants which had been treated with 63 ppm of the active compounds I-10, I-11, I-13, I-14 or I-17 were at most 30% infected and the untreated plants were likewise 90% infected.

### Comparative Example 2

### Activity Against peronospora of Grapevines Caused by Plasmopara viticola, 7 Day Protective Treatment

Leaves of potted vines were sprayed to runoff point with an aqueous suspension having the concentration of active compounds stated below. To be able to assess the persistency of the substances, the plants were, after the spray coating had dried on, placed in a greenhouse for 7 days. Only then were the leaves inoculated with an aqueous zoospore suspension of Plasmopara viticola. The vines were then initially placed in a water-vapor-saturated chamber at 24° C. for 48 hours and then in a greenhouse at temperatures between 20 and 30° C. for 5 days. After this time, the plants were once more placed in a humid chamber to accelerate the eruption of sporangiospores. The extent of the development of the infection on the undersides of the leaves was then determined visually.

In this test, the plants which had been treated with 250 ppm of the active compound I-3 or 1-5 showed an infection of not more than 10%, whereas the plants which had been treated with 250 ppm of the comparative active compound A were 70% infected and the untreated plants were 90% infected.

### Use Example 3

### Activity Against peronospora of Grapevines Caused by Plasmopara viticola

Leaves of potted vines were sprayed to runoff point with an aqueous suspension having the concentration of active compounds stated below. The next day, the undersides of the leaves were inoculated with an aqueous sporangia suspension of Plasmopara viticola. The vines were then initially placed in a water-vapor-saturated chamber at 24° C. for 48 hours and then in a greenhouse at temperatures between 20° C. and 30° C. for 5 days. After this time, the plants were once more placed in a humid chamber for 16 hours to accelerate the eruption of sporangiospores. The extent of the development of the infection on the undersides of the leaves was then determined visually.

In this test, the plants which had been treated with 250 ppm of the active compound I-1, I-2, I-3, I-5, I-6 or I-9 showed no infection, whereas the untreated plants were 90% infected.

### Use Example 4

### Activity Against Late Blight on Tomatoes Caused by Phytophthora infestans, Protective Treatment

Leaves of potted tomato plants were sprayed to runoff point with an aqueous suspension having the concentration of active compounds stated below. The next day, the leaves were infected with an aqueous sporangia suspension of Phytophthora infestans. The plants were then placed in a water-vapor-saturated chamber at temperatures between 18 and 20° C. After 6 days, the late blight on the untreated, but infected control plants had developed to such an extent that the infection could be determined visually in %.

In this test, the plants which had been treated with 250 ppm of the active compound I-5 or I-6 showed an infection of at most 10%, whereas the untreated plants were 90% infected.

### Use Example 5

### Activity Against Late Blight on Tomatoes Caused by Phytophthora infestans, 3 day Protective Treatment

Leaves of potted tomato plants were sprayed to runoff point with an aqueous suspension having the concentration of active compounds stated below. After three days, the leaves were infected with an aqueous sporangia suspension of Phytophthora infestans. The plants were then placed in a water-vapor-saturated chamber at temperatures between 18 and 20° C. After 6 days, the late blight on the untreated, but infected control plants had developed to such an extent that the infection could be determined visually in %.

In this test, the plants which had been treated with 250 ppm of the active compound I-1 or I-2 showed an infection of at most 5%, whereas the untreated plants were 90% infected.

1. The use of an 5-alkyl-6-phenylalkyl-7-aminoazolopyrimidine of the formula I 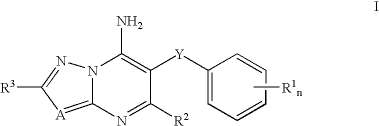
in which the substituents are as defined below:
Y is C₁-C₆-alkylene, C₂-C₆-alkenylene or C₂-C₆-alkynylene, optionally substituted by 1 to 4 C₁-C₆-alkyl groups;
R¹ is halogen, cyano, nitro, hydroxyl, mercapto, C₁-C₆-alkyl, C₁-C₄-haloalkyl, C₂-C₆-alkenyl, C₃-C₆-cycloalkyl, C₃-C₆-cycloalkenyl, C₁-C₆-alkoxy, C₁-C₆-haloalkoxy, C₂-C₆-alkenyloxy, C₃-C₆-alkynyloxy, C₁-C₆-alkylthio, NR^{A}R^{B}, C₁-C₆-alkylcarbonyl, phenyl, naphthyl, or a five- or six-membered saturated, partially unsaturated or aromatic heterocycle which contains one to four heteroatoms from the group consisting of O, N and S;
R^{A}, R^{B} are hydrogen, C₁-C₆-alkyl or C₁-C₆-alkylcarbonyl;
n is zero, 1, 2, 3 or 4;
R² is C₂-C₆-alkyl, C₂-C₄-alkenyl, C₃-C₆-cycloalkyl, C₁-C₁₂-alkoxy-C₁-C₁₂-alkyl or C₁-C₁₂-alkylthio-C₁-C₁₂-alkyl;
R³ is hydrogen, halogen, cyano, NRARB, hydroxyl, mercapto, C₂-C₆-alkyl, C₁-C₆-haloalkyl, C₃-C₈-cycloalkyl, C₁-C₆-alkoxy, C₁-C₆-alkylthio, C₃-C₈-cycloalkoxy, C₃-C₈-cycloalkylthio, carboxyl, formyl, C₁-C₁₀-alkylcarbonyl, C₁-C₁₀-alkoxycarbonyl, C₂-C₁₀-alkenyloxycarbonyl, C₂-C₁₀-alkynyloxycarbonyl, phenyl, phenoxy, phenylthio, benzyloxy, benzylthio, C₁-C₆-alkyl-S(O)ₘ—;
m is 0, 1 or 2;
A is N or C—R^{a};
R^{a} is hydrogen or C₁-C₆-alkyl;
where the carbon atoms in Y, R¹, R², R³ and R^{a} may carry one to three groups R^{b}:
R^{b} is halogen, cyano, nitro, hydroxyl, C₃-C₆-cycloalkyl, C₁-C₆-alkoxy, C₁-C₆-alkylthio or NR^{A}R^{B};
for controlling phytopathogenic harmful fungi.

2. The compound of the formula I according to claim 1 in which A is a nitrogen atom.

3. The compound of the formula I according to claim 1 in which R² is ethyl.

4. The compound of the formula I according to claim 1 in which R¹ is not phenyl.

5. The compound of the formula I according to claim 1 in which R³ is hydrogen.

6. The compound of the formula I according to claim 1 in which A is C—R^{a}, where R^{a} is C₁-C₆-alkyl which may be substituted by one to three groups R^{b} according to claim 1.

7. The compound of the formula I according to claim 1 in which the substituents are as defined below:
Y is C₁-C₆-alkylene, optionally substituted by 1 to 4 C₁-C₆-alkyl groups;
R¹ is halogen, C₁-C₆-alkyl or halomethyl;
n is zero, 1, 2 or 3;
R² is C₂-C₆-alkyl, C₃-C₆-cycloalkyl or C₁-C₁₂-alkoxy-C₁-C₁₂-alkyl;
R³ is hydrogen, NH₂ or C₁-C₆-alkyl;
A is N or C—R^{c}C;
R^{c} is hydrogen or C₁-C₆-alkyl.

8. A process for preparing the compound of the formula I according to claim 1, wherein a β-ketoester of the formula II 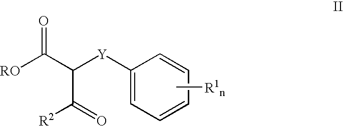
in which R is C₁-C₄-alkyl is reacted with a-aminoazole of the formula III 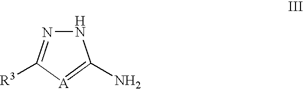
to give a 7-hydroxyazolopyrimidine of the formula IV 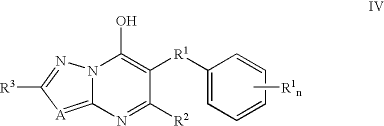
which is halogenated to give a compound of the formula V 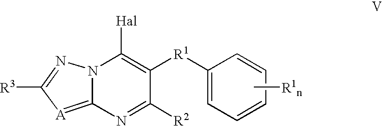
in which Hal is chlorine or bromine and V is reacted with ammonia.

9. A compound of the formula IV or V according to claim 8.

10. A process for preparing the compound of the formula I according to claim 2, wherein an acylcyanide of the formula VI 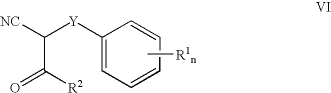
is reacted with a-aminoazole of the formula III according to claim 8.

11. A composition comprising a solid or liquid carrier and the compound of the formula I according to claim 2.

12. The composition according to claim 11 comprising a further active compound.

13. Seed comprising the compound of the formula I according to claim 1 in amounts of 1 to 1000 g per 100 kg.

14. A method for controlling phytopathogenic harmful fungi, wherein the fungi or the materials, plants, soil or seed to be protected against fungal attack are treated with an effective amount of the compound of the formula I according to claim 2.
